(19) United States
(12) Patent Application Publication
(10) Pub. No.: US 2014/0342008 A1
(43) Pub. Date: Nov. 20, 2014

(54) PERSONAL CARE SUBSTANCES HAVING CERTAIN MINERAL AND ORGANIC BASED CONSTITUENT MATERIALS

(71) Applicants: Tiffany Andersen, Las Vegas, NV (US); Robert Allen Barlett, Tulalip, WA (US)

(72) Inventors: Tiffany Andersen, Las Vegas, NV (US); Robert Allen Barlett, Tulalip, WA (US)

(21) Appl. No.: 14/276,752

(22) Filed: May 13, 2014

Related U.S. Application Data
(63) Continuation-in-part of application No. 14/276,709, filed on May 13, 2014.
(60) Provisional application No. 61/823,227, filed on May 14, 2013.

(51) Int. Cl. A61K 8/98; A61K 8/92; A61Q 19/00; A61K 8/19

(52) U.S. Cl. CPC A61K 8/982; A61K 8/19; A61K 8/92; A61Q 19/00; A61K 2800/10
USPC 424/520

(57) ABSTRACT

Skin care creams and atomizable liquids, based on mineral based ingredients such as those in gemstones and gold, and organically based ingredients such as organic solvents, eggs, and others. The personal care substances are in fluid form, including a carrier fluid together with active ingredients. The final fluids may be topically applied, or may be used as additives to other personal care substances.

### REFERENCE TO RELATED APPLICATION

This application is a continuation-in-part application of application Ser. No. 14/276,709, filed on May 13, 2014, claims the benefit of U.S. Provisional Application No. 61/823,227, filed on May 14, 2013.

### FIELD OF THE INVENTION

The present invention relates to personal care substances for topical application to the skin.

### BACKGROUND OF THE INVENTION

Personal care substances have long been sought by consumers, and have found an enthusiastic market. These personal care substances may be applied externally to the skin. While many personal care substances have been developed relatively recently, others which rely on historically known ingredients may also be developed.

### SUMMARY OF THE INVENTION

The present invention provides personal care substances which utilize certain specific ingredients which have traditionally or historically been utilized. Accordingly, the invention contemplates processes for preparing personal care substances, based on mineral based ingredients such as those in gemstones and gold, and organically based ingredients such as organic solvents, eggs, and others. The personal care substances are in fluid form, including a carrier fluid together with active ingredients. The final fluids may be topically applied, or may be used as additives to other personal care substances.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification.

### DETAILED DESCRIPTION

According to at least one aspect of the invention, there is shown a skin care product comprising a fluid vehicle; egg oil, cell salts, and ozone oil; and at least one of gold, silver, copper, diamonds, garnets, pearls, egg oil, ozonated oil, and cell salts. Other active ingredients may include a large array of botanicals, peptides, stem cells, and ecologically friendly preservatives.

The product may take the form of a cream, or alternatively, an atomizable liquid to be sprayed onto the body. A cream is a fluent material of such viscosity as to flow responsive to forces such as finger pressure, but to flow almost imperceptibly if acted on only by gravity.

The cream, which may be a face cream intended for application to the skin of the face, may comprise the following active ingredients: matrixyl 3000 (3-8%) (percentages shown herein are weight percentages); apple stem cells (2-5%); alpha arbutin (0.2-2%); and NatuPres (2%).

The face cream may have a vehicle comprising purified water, Aloe Barbadensis (Aloe) Leaf Oil, Aloe Barbadensis (Aloe) Leaf Juice, Palm Oil Based Emulsifier, Vegetable Wax, Kosher Vegetable Glycerin, Vegetable derived humectants, Vegetable Silicone, Coconut Oil derived emulsifier, Acmella Oloracea extract, White Tea Leaf Extract, Cucumber Extract, Lentinus Edodes Extract, Hydrolyzed Cucurbito Pepo (Pumpkin) Seedcake, Prunus Amygdalus Dulcis (Sweet Almond) Seed Extract, Matrixyl™ 3000 (Palmitoyl Oligopeptide and Palmitoyl Tetrapeptide-7), PhytoCellTech™ (Malus Domestica Fruit Cell Culture Extract), Phylderm Vegetal CII™ (Fractionated Rice Extract Di and Tri Peptides), Vitis Vinifera (Grape) Seed Extract, Blue Algae Extract, Palmaria Palmata (Sea Parsley) Extract, Spirulina Platensis (Spirulina) Extract, Collagen Peptide, Juglanis Regia (Walnut Oligopeptide) Seed Extract, Glutathione, Superoxidase Dismutatase, Co-Q10 (Ubiquinone) Enzyme, Phospholipids, Tocopheryl Acetate (Vitamin E Acetate), Yeast Beta Glucan, Leuconostoc/Radish Root Ferment Filtrate, Lonicera Japonica (Honeysuckle) Flower Extract, Populus Tremuloides Bark Extract, and Fragrance.

Another product, which for various reasons is called Tri-peptide-C Lightening Serum, may include purified water (Aqua), Chondrus Chrispus (Carageenan), Grapeseed extract (Vitis Vinifera (Grape) Seed Extract, Punica Ganatum (Pomagranate) Fruit Extract, Magnesium Ascorbyl Phosphate, Juglanis Regia (Walnut Oligopeptide) Seed Extract, Phylderm Vegetal CII™ (Fractionated Rice Extract Di and Tri Peptides), Vaccinium Macrocarpon (Cranberry) Fruit Extract, Vaccinium Augustifolium (Blueberry) Fruit Extract, Sodium Hyaluronate (Hyaluronic Acid), Panthenol (Pro-Vitamin B5), Kosher Vegetable Glycerin, Leuconostoc/Radish Root Ferment Filtrate, Lonicera Japonica (Honeysuckle) Flower Extract, Populus Tremuloides Bark Extract.

Another cream product, which will be for various reasons called Hyaluronic Acid Peptide Serum, may include Matrixyl 3000 (8%), Adenine PBC (5%), Relistase (4%), Stem Cells (5%), NatuPres (2%), Collagen Peptide (3%), and Alpha Arbutin (2%).

With these additional peptides and actives for a quantity of the cream product suitable for retail sale, such as 1920 gms (64 oz), there will be added 15-20 drops of liquid gold and or diamonds, garnets, pearls or cell salts to the cream product to make different serums Any of these serums may include pure gold leaf (99.99% pure).

The fluent vehicle of the cream product may include purified water, Sodium Hyaluronate (Hyaluronic Acid), Matrixyl™ (Palmitoyl Oligopeptide & Palmitoyl Tetrapeptide-7), Adenine PBC™ (Tripeptide-1), Relistase™ (Acetylarginyltrytophyl Diphenylglycene Tripeptide), PhytoCellTech™ (Malus Domestica Fruit Cell Culture Extract, Collagen peptide, Kosher Vegetable Glycerin, Leuconostoc/Radish Root Ferment Filtrate, Lonicera Japonica (Honeysuckle) Flower Extract, and Populus Tremuloides Bark Extract.

Gold oil may be present in the cream in a range of 5 to 30 drops per 1 oz of cream. The same ratio would apply for any skin cream product according to the present invention formulated with silver, copper, diamonds, garnets, or pearls. Egg oil may be present in a range of 250 drops or less per 1 oz cream or oil serum. The same ratio may apply for ozonated oil and cell salts.

A cream intended for the eyelids and other facial skin may include gold, egg oil, cell salts, silver, copper, diamonds, garnets, pearls and ozone oil to this formula but is susceptible to certain different constituencies. For example, only one of the silver, copper, diamonds, garnets, and pearls may be present.

An atomizable liquid for being dispensed as a mist to the face, for example, may include egg oil or cell salts, and optionally, at least one of gold oil, diamonds, garnets, pearls, and optionally, pure gold.

A liquid vehicle may include purified alkaline water, gold oil, Aloe Barbadensis Leaf Juice, Ozonated plant oil, pure Gold, Green Tea Extract, Gogi Berry Extract, Medical Grade Hyaluronic Acid, and Food Grade Essential Oil.

A serum which for various reasons will be called Golden Radiance Serums may include gold oil and/or diamonds, garnets, pearls, egg oil or cell salts, and optionally, a very small amount of pure gold.

A fluid vehicle may include grapeseed oil, 60%, ozone oil, 10%, blackberry oil, 5%, pomegranate oil, 5%, and red raspberry oil, 5%.

All constituent materials will be of food grade, where such a standard exists in the industry.

The above described products may oppose visible aging of the skin, and may help promote new skin cells and collagen production, thereby reducing appearance of fine lines, wrinkles, pigmentation and helping with acne and other inflammatory skin conditions.

Fluids containing essential constituent materials of gems, such as diamond, garnet, pearl, and others may be prepared as follows. A quantity of gems sufficient for an extraction is crushed and calcined. Some of the previously prepared solvent is added after cooling the crushed and calcined gem material to about room temperature. The mixture is circulated in a suitable vessel such as a pelican. For the purposes of this application, a pelican is a circulatory distillation vessel with two side-arms feeding condensed vapors back into the body. The pelican is so-called as it resembles a pelican pecking at its breast to feed its hatchlings. When a tincture forms, the tincture is removed, and the solvent is removed from the tincture by distillation. A thicker fluid, appearing as an oil, is left behind.

Fluids such as egg oil may be prepared as follows. The following example describes the preparation of an oil derived from egg yolks using supercritical fluid extraction. One hundred twenty eggs are dehydrated to obtain powdered yolk. This is done by hard boiling the eggs, recovering the yolks, dehydrating for several hours until all water is removed, and comminuting them into a powder. The dehydrated egg yolk powder is placed into a glass tube. A supercritical fluid, such as carbon dioxide or butane, is injected into the top of the glass tube. An oil or fluid will evacuate from the bottom and is collected in a collection dish. The oil can be further refined until all solids are removed.

Fluids containing metal salts may be prepared as follows. Spagyric mineral salts may be combined with certain metal acetates to generate a useful additive. Distilled metal acetate is used to extract a fluid bearing comminuted crystals. A spagyric method is one in which essential components of a material are first separated then purified by appropriate techniques and finally reunited into a new homogenous whole. The resulting product is held to have enhanced effectiveness compared to the crude or merely chemically purified substance. As an example of the process, a preparation of minerals called Schussler Cell Salts will be described. The Schussler Cell Salts include potassium phosphate, sodium sulfate, potassium chloride, calcium fluoride, magnesium phosphate, potassium sulfate, sodium phosphate, calcium sulfate, silicon dioxide, calcium phosphate, sodium chloride, and iron phosphate. These salts are required in USP of NF grade. In addition the metal acetate of the parent metal of the particular salt is also required. For example there are three salts of calcium listed above, therefore calcium acetate is required to produce all three. In all, five acetates are required to produce the twelve salts, including potassium acetate, sodium acetate, magnesium acetate, calcium acetate, and iron acetate.

A fluid which for various reasons is called Gold oil may be prepared as follows. The following exemplary process describes the preparation of an oily product through the extraction of a pure gold precipitate with a specially prepared solvent mainly consisting of ethyl alcohol to which a sublimated salt has been added. For various reasons, the resulting product will be called “the oil of gold”, and represents a class of materials of medical interest called “potable gold”.

The process utilizes gold, with a preferred source being gold ore that has not been melted (e.g., placer mined nuggets or flakes). Pure gold which has been refined by fire can also be used. Gold of lesser karat value than 24 must be refined, with refinement through the use of antimony preferred.

A solvent used in this process is prepared by sublimating sal ammoniac (NH₄Cl) several times until a yellow or orange crystal forms. The preferred salt is sal ammoniac but certain other salts may be substituted. Pure gold is dissolved into aqua regia and the resulting solution is filtered through a 40 micron glass frit. The filtered liquid is slowly evaporated on a low heat until it crystallizes. The crystals are then re-crystallized from a 1M solution of hydrochloric acid and then re-crystallized from distilled water. The resulting deep golden orange crystals of hydrogen tetrachloroaurate are then gently surface dried and stored in an airtight glass container kept cool and out of direct light. Care must be taken as the crystals are very hygroscopic and corrosive and will burn and stain the skin. This is then precipitated out into a purple powder with a variety of compounds such as tin chloride, or potassium carbonate, washed several times and filtered with a glass filter in a Buchner funnel.

A solvent is prepared by sublimating Sal Ammoniac (NH₄Cl) until yellow, orange, or red crystals form. These crystals are collected and placed in a beaker that can be hermetically sealed. The beaker is filled with dehydrated alcohol (e.g., reacted with potassium carbonate crystals) and the entire contents are circulated for an extended period of time. The contents are then hermetically sealed. Once the extraction is complete from the circulation it is distilled three to seven times, continually pouring the liquid back into the retort together with the salt that didn't carry over. Once finished, the distillate is collected and saved in a cool place for use in extracting the oil from the precipitate.

To extract gold precipitate and solvent, a measured amount of prepared solvent is placed into a glass borosilicate flask to which is added a weighed quantity of the prepared gold precipitate (quantities are discussed hereinafter). The gold powder will slowly release its oil at a low to moderate temperature in the prepared solvent over several months. The reaction vessel is sealed and allowed to stand at room temperature for at least one month in the dark. A darkening of the solution will increase over time.

After the reaction time, the remaining oil is further refined by a variety of processes including severe temperature fluctuations, fractional distillation and extraction with a variety of alcohols, specifically ether, the prepared solvent, and all the alcohols can be reused by simply distilling and collecting. The final oil is then placed in 96% grain alcohol and used internally at a dose of 1-3 drops, or directly on the skin.

Oil of gold may be prepared through the catalytic effect of gold chloride on a specially prepared solvent mainly consisting of acetone. For various reasons, the resulting product has been labeled as “The Oil of Gold”. Pure gold is dissolved into aqua regia and the resulting solution is filtered through a 40 micron glass frit. The filtered liquid is slowly evaporated on a low heat until it crystallizes. The crystals are then recrystallized from a 1M solution of hydrochloric acid and then recrystallized from distilled water. The resulting deep golden orange crystals of Hydrogen tetrachloroaurate are then gently surface dried and stored in an airtight glass container kept cool and out of direct light. They are very hygroscopic and corrosive and will burn and stain the skin.

The solvent is prepared by the dry distillation of one of the selected metal acetates gradually up to a maximum temperature of 700 degrees Celsius. Special attention is paid to the cooling system used to condense the vapor produced in the distillation. The collected distillate is placed into a fractional distillation train and the fraction distilling over up to 56 degrees Celsius is collected. The resulting clear, volatile liquid is preserved in a sealed glass container stored in a cool place.

A measured amount of prepared solvent is placed into a glass flask to which is added a weighed quantity of the prepared gold crystals. The gold crystals will dissolve with a darkening of the solution which increases over time. The reaction vessel is sealed and allowed to stand at room temperature for at least one month in the dark.

After the reaction time, the solution will contain a precipitate of metallic gold. This may also appear in the form of metallic gold foil coating the glass vessel. The now dark amber red solution is filtered away from the gold precipitate and is sealed in a clean glass vessel for aging. This solution of “oil of gold” can be used in various products neat, or may be incorporated into creams and atomizable liquids such as those described herein.

Although there have been no modern clinical trials, benefits that have been reported after taking a few drops of the gold oil every few days. Historically, Oil of Gold has been applied as a treatment of rheumatism, arthritis, cancer, syphilis, uremia and multiple sclerosis.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. Topical skin care product, comprising:
a fluid vehicle;
egg oil, cell salts, and ozone oil; and
at least one of gold, silver, copper, diamonds, garnets, pearls, egg oil, ozonated oil, and cell salts.

2. The topical skin care product of claim 1, further comprising gold oil in a range of 5 to 30 drops per fluid ounce of fluid vehicle.

3. The topical skin care product of claim 1, wherein the topical skin care product is a facial cream usable on the face and eyes.

4. The topical skin care product of claim 1, wherein the topical skin care product is an atomizable fluid for application to the face.
